(19) United States
(12) Patent Application Publication
(10) Pub. No.: US 2012/0225422 A1
(43) Pub. Date: Sep. 6, 2012

(54) METHOD AND DEVICE EMPLOYING A NON-RECEPTOR LIGAND INTERACTION WITH NANOPARTICLES OR OTHER SOLID PHASE FOLLOWED BY SPECIFIC DETECTION

(75) Inventor: Ramakrishna Bharadwaj Boyanapalli, Westminster, CO (US)

(73) Assignee: RoMonics, LLC, Boulder, CO (US)

(21) Appl. No.: 13/408,358

(22) Filed: Feb. 29, 2012

Related U.S. Application Data
(60) Provisional application No. 61/449,061, filed on Mar. 3, 2011.

(51) Int. Cl. G01N 33/569; B01L 3/00; G01N 33/558; B82Y 15/00

(52) U.S. Cl. 435/5; 436/514; 435/6.1; 422/500; 977/700; 977/920

(57) ABSTRACT

A method and apparatus for conducting specific binding assays are claimed. This invention relates to an initial non-receptor ligand interaction with nanoparticles or other solid phases followed by specific detection methods used for detecting biological, chemical or environmental entities. This invention employs an on-device ligand attachment to nanoparticles or other solid phase followed by the specific capture in discrete zones on the device. The ligand-bound complexes assemble on the ligand specific capture zone leading to a visible diagnostic result, if colored solid phases are employed.

### PRIORITY AND RELATED ART

### U.S. Patent Documents

U.S. Pat. No. 7,393,697 B2. Charlton, 2008
U.S. Pat. No. 6,541,277 B1. Kang et al., 2003
U.S. Pat. No. 6,534,320 B2. Ching et al., 2003
U.S. Pat. No. 6,485,982 B1. Charlton et al., 2002
U.S. Pat. No. 6,352,862 B1. Davis et al., 2002
U.S. Pat. No. 5,591,645. Rosenstein et al. 1997
U.S. Pat. No. 4,743,560. Campbell et al. 1988

### BACKGROUND OF THE INVENTION

1. Field of the invention

This invention relates to an immunoassay used for detecting biological, chemical, or environmental entities. Several assay systems use colloidal or particulate conjugates and nitrocellulose membranes, each with immobilized antigen and/or antibody. Many ligands, however, are not easily detected with a typical assay system (e.g. lateral flow and flow through assay systems) due to their size, paucity of available epitopes, and/or other factors. This invention employs an on-device ligand attachment to nanoparticles followed by detection of the ligand in a discrete zone on the device. When colored nanoparticles are used, the ligand-bound nanoparticles assemble on the ligand specific capture zone leading to a visible result. This method of on-device attachment of the ligands to reactive entities has never been previously reported.

2. State of the art

Several approaches have been developed for detection of various types of molecules of interest. Some of these methods include Enzyme-linked Immunoassay (ELISA), immunochromatographic devices (lateral flow), and flow-through devices. In some of these assays the sample flows laterally and vertically respectively through a microporous membrane from the zone of application to a region on the membrane where a specific capture reagent is present. The analyte of interest can be directly visualized at the capture reagent line or zone. These approaches have been used to detect a variety of analytes, including antigens and antibodies. Other testing methods include nucleic acid amplification techniques such as polymerase chain reaction (PCR), RT-PCR, nucleic acid sequence-based amplification (NASBA), transcription-mediated amplification (TMA), loop-mediated isothermal amplification (LAMP), helicase-dependent amplification (had), etc. In addition, diagnostic platforms have also been used to detect tumor markers, cardiac markers, and drugs of abuse.

The above-mentioned approaches have limitations since they generally rely on at least two antibodies to form a sandwich. Some ligands, however, have fewer epitopes or binding sites than others and thus the selection of appropriate pair of receptors, antibodies, or binding molecules may present a diagnostic challenge.

The present invention addresses the above-mentioned limitations by providing a method for a non-receptor mediated binding followed by a specific capture.

### DEFINITIONS

Analyte or Ligand: Any substance being analyzed
Colloid: A substance microscopically dispersed evenly throughout another one
Complex: Resultant crystalline or aggregated reactants
Conjugate: The union of soluble or insoluble entities
Flow through: Membrane-based flow through system for analyte detection
Lateral flow: Membrane-based immunochromatographic test device used at point-of-care
Microparticle: The term is usually used for particles ranging 300 to 700 nm. The terms “latex beads”, “uniform microspheres”, and “microparticles” are sometimes used interchangeably
Nanoparticle: A microscopic particle whose size is measured in nm. The term is usually reserved for particles with dimensions less than 100 nm
Nucleic acid: Group of complex compounds, composed of purines, pyrimidines, carbohydrates, and phosphoric acid. Nucleic acids in the form of DNA and RNA control cellular function and heredity

### SUMMARY OF INVENTION

The invention involves modification of a typical lateral flow immunoassay device by combining real-time ligand attachment to nanoparticles (either by passive adsorption or covalent coupling), any other modifications to ligands (gene amplification, ligand tagging, blocking, etc.), and capture of the ligand bound to the nanoparticles at the ligand specific test line on a single device. The ligand in this diagnostic device may be any biological, chemical, or environmental analyte. The modification of lateral flow device along with binding and detection on a single embodiment makes these disposable, point-of-care diagnostic tests simple, affordable, and rapid. This modification which exploits non-specific capture of a target molecule followed by specific detection on a diagnostic device is a unique diagnostic approach for the rapid diagnosis of disease.

The invention also relates to any nanoparticle based device containing capable of binding a target of interest in a non receptor-ligand fashion followed by specific capture on another solid phase

In one approach, colored reactive nanoparticles can bind molecules including the target ligands present in the sample at the target entry zone. The present invention allows for the continuous attachment of a target to a surface in a non receptor-ligand fashion before specific capture. The binding of the ligands to the active nanoparticles can be facilitated through various forces.

Examples of attachment forces are hydrophobic, covalent coupling, non-covalent binding, hydrogen bonding, Van der Waals, and ionic interaction. After binding, the ligand-nanoparticle complexes, when applied to a porous membrane such as nitrocellulose, will migrate to the capture zone. At the capture zone, the ligand-nanoparticle complexes accumulate at the test zone by binding to the ligand specific receptors impregnated in the nitrocellulose membrane. The accumulated ligand-nanoparticle complexes appear as a visible color, reflecting the assembly of nanoparticles. When the sample is devoid of ligand of interest, the nanoparticles migrate to the capture zone without binding to the receptors at the capture zone thus resulting in no visible line that can be easily read as a negative reaction.

The target entry zone may contain colored nanoparticles called control nanoparticles that are of a different color from the reactive nanoparticles so that the colors can be easily distinguished. These control nanoparticles have specific molecules attached and can flow with the bound target without impacting their flow characteristics. In the case of lateral flow, the control nanoparticles migrate beyond the test line in the capture zone and accumulate at the control line by binding to receptors specific for the control nanoparticles that are impregnated in the nitrocellulose membrane. For the flow through device, they will assemble on the zone containing a specific receptor. The accumulated control nanoparticles appear as a visible control line.

Control nanoparticles may be coated with biotin and the control capture zone may be comprised of NeutrAvidin™. This practice is not limited to colored nanoparticles or microporous media. It will be appreciated by those skilled in the art that the conjugate may be in multiple forms such as particles of various sizes and shapes, metal sols, colored polystyrene uniform microspheres, etc.

Although the preferred embodiment of the invention can be read unaided, it can also be used with a variety of approaches such as chemiluminescence, fluorescence, and other chromogenic substrates when enzymes are employed. These can all be used with instruments.

### BRIEF DESCRIPTION OF DRAWINGS

FIG. 1. Modified lateral flow immunoassay with details of the target and capture zones

FIG. 2. Testing target entity on the modified lateral flow immunoassay setup

FIG. 3. This figure shows a view of the positive result and the control result

FIG. 4. The figure explains passive adsorption of ligand to an active nanoparticle

FIG. 5. Depiction of a device and for the primary interaction of ligand to the solid phase

FIG. 6. Flow-through device with specific capture entities for selecting complexes

FIG. 7. Negative and positive reactions on the same solid phase device

### DETAILED DESCRIPTION OF PREFERRED EMBODIMENT

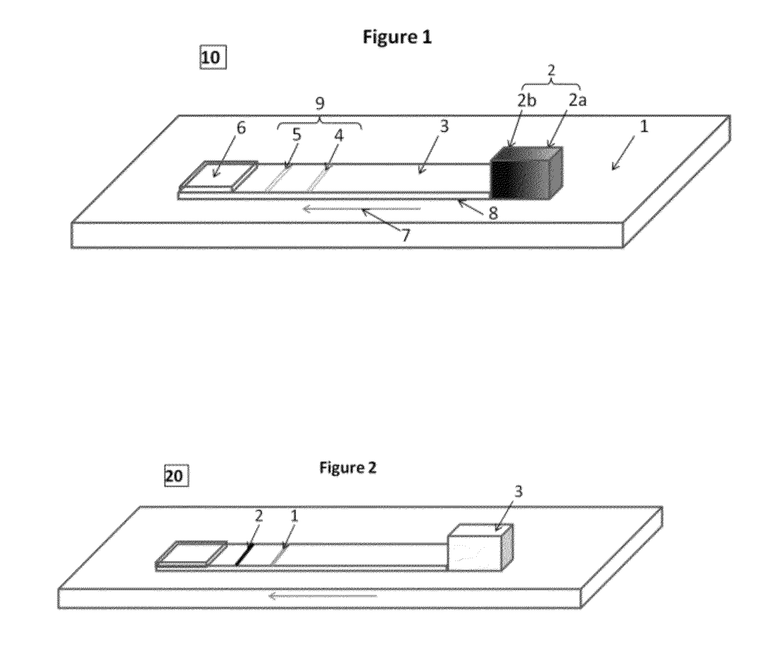

This modified lateral flow immunoassay setup (10) shows the details of the target zone as a chamber or fabric containing blue colored active and red colored non-reactive nanoparticles and capture zone with both test line and control line. Flow direction of the nanoparticle complexes on the porous membrane such as nitrocellulose is shown as an arrow towards capture zone (FIG. 1).

When the target entity is tested on the modified lateral flow immunoassay setup, both red control nanoparticles and blue target ligand-nanoparticle complexes migrate from the target zone to the capture zone (20). The migrated blue target ligand-nanoparticle complexes accumulate at the test line in visible blue color line (1) and the migrated red control nanoparticles accumulate at the control line in visible red color line (2). This reaction is determined as a positive diagnostic result for the target ligand (FIG. 2).

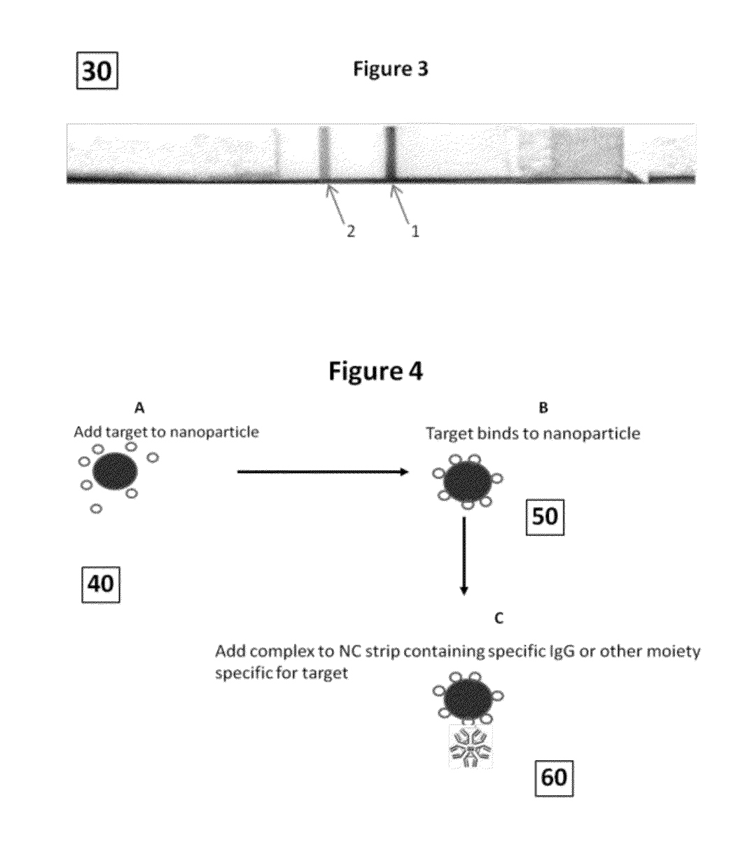

FIG. 3 depicts the positive result explained in (20), where the migrated blue target ligand-nanoparticle complexes accumulate at the test line in visible blue color line and the migrated red control nanoparticles accumulated at the control line in visible red color line. This reaction is determined as a positive diagnostic result for the target ligand (30)

An illustration of passive adsorption of ligand to an active nanoparticle is shown in FIG. 4.

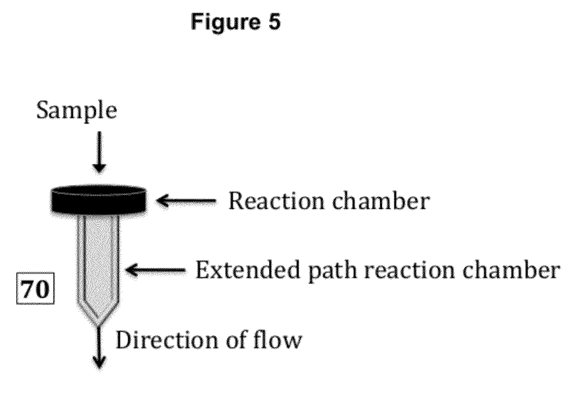

When a target entity with ligand of interest comes in close proximity of hydrophobic or reactive regions of the blue active nanoparticle (40), the non-polar or aromatic regions of the ligand adsorbs strongly to these regions of the nanoparticles (50). The ligand-nanoparticle complexes (B) migrate along the porous membrane such as nitrocellulose to the capture zone as shown in FIG. 2 and FIG. 3, and are arrested at the test line with ligand specific receptors (60). Different complexes may be read in discrete visible lines. The depiction of a device and chamber that allows for the primary interaction of ligand to the solid phase (70) is shown in FIG. 5. Note an extension path that may be used, if necessary, for further separation of bound and free reagents. This separation can be achieved by a variety of well-established chromatographic techniques (e.g. ion exchange, gel filtration, and affinity methods).

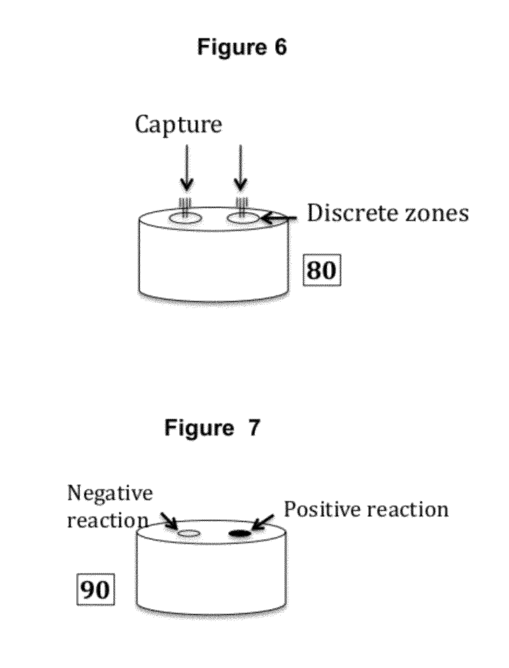

FIG. 6 depicts an example of a flow-through device with specific capture entities for selecting nanoparticle-ligand complexes (80). The complexes will bind to the specific capture when they pass through the solid phase device.

A depiction of both negative and positive reactions on the same solid phase device is shown in FIG. 7. A negative reaction where nanoparticle-ligand complexes did not bind to the unmatched receptor is shown as an empty circle. A positive reaction where nanoparticle-ligand complexes bind to the matched receptor is shown as a filled circle (90).

The invention is a modified flow device, where attachment of nanoparticles to the target ligand is achieved in the zone on the device followed by migration of the target ligand-nanoparticle complexes at another zone on the device where specific capture occurs. The initial attachment is not due to a receptor-ligand interaction or antigen antibody interaction. The attachment of ligand and active nanoparticles can be passive adsorption, at hydrophobic regions of the nanoparticles, or covalent coupling of ligand specific functional group/s on the active nanoparticles, or via non-covalent binding, hydrogen bonding, ionic interaction, covalent binding, or any reaction leading to the formation of target ligand-nanoparticle complexes. After these complexes are formed, a blocking buffer containing a protein such as casein, bovine serum albumin, synthetic long chain peptides, synthetic short chain peptides, etc., may be added to inactivate the unreacted nanoparticles. In the same chamber, a ligand can be modified by the attachment of other tagging molecules. In addition, immobilized nucleic acid can be amplified using molecular amplification techniques. Samples may be contained in various diluents and buffers. Buffers may consist of a wide range of pHs and may be comprised of carbonate as in the case of 20 mM Na₂CO₃/NaHCO₃, pH 9.6, phosphate such as 10 mM Na₂HPO₄, MES, HEPES, Borate, Acetate, and others. These may or may not contain salts such as sodium chloride. Commonly used are 10 mM phosphate buffered saline (PBS), pH 7.2 and 50 mM HEPES, pH 8.0. Blocking agents may include amino acids, peptides, and proteins. Other additives may include sugars, surfactants, synthetic polymers, etc. Examples of approaches that can be used with the modified lateral flow immunoassay device include methods for testing deoxyribonucleic acid (DNA), ribonucleic acid (RNA), aptamers, oligonucleotides, polymerase chain reaction (PCR) products, reverse transcriptase PCR, real time quantitative PCR, and products derived from isothermal amplification systems such as nucleic acid sequence-based amplification (NASBA) and loop mediated amplification (LAMP), cloning, gene targeting, high throughput screening (HTS), capillary electrophoresis, nucleic acid sequence analysis, nucleic acid labeling and detection, gene expression analysis, single-nucleotide (SNP) analysis, recombinant DNA analysis, RNAi. In the case of small peptides, glycoproteins, and various proteomic compounds, the art can be used for receptors (e.g. β-Adrenergic, T and B Cell Ligands, Adenosine), enzymes, Interleukins, Complement components, components of hemostasis, hormones (e.g. reproductive endocrine hormones: hCG, LH, FSH), activator and repressor proteins, phage display peptide libraries, Prions, Penicillin-binding proteins, cluster of differentiation (CD) molecules, Chemotaxins, individual amino acids, markers used to study cardiac health, such as Troponin T and I, proBNP and homocysteine. It may also be applied in serology, as in the cases of IgG, IgA, IgM, Ig profiling. Other applications include protein-protein interactions, such as transcription factors interactions, signaling molecule interactions, blood coagulation factors interactions, etc., and for cell signaling, such as MAP kinase pathway, RAS pathway, NFkB pathway, NFAT pathway, etc. In addition, the method can be used for biological molecules that are up or down regulated in various cardiovascular diseases. Also relevant, are molecules that are up or down regulated in various respiratory diseases, tumors and carcinomas and various neurological diseases. Others include molecules that are up or down regulated in various kidney diseases, etc. Other molecules include lectins and other cell surface receptors, polymyxin-LPS, antioxidants, Neopterin, whole organisms, such as viruses, bacteria, and fungi. Also applicable are substance abuse, such as alcohol, amphetamines, barbiturates, benzodiazepines, cocaine, methaqualone and opioids.

Several sample types or specimens can be used with this system. These include whole blood, plasma, serum, urine, stool, water, food extracts, dirt extracts and chemicals.

### EXAMPLES

### Example 1

### Construction of a Device Based on Non-Specific Attachment in the Initial Step

A virus-detecting device was constructed. Essentially, a virus was amplified in a specific host and the progeny adsorbed to nanoparticles non-specifically. The nanoparticle-target complexes were allowed to migrate on the nitrocellulose membrane to the virus specific capture zone.

After an incubation period, 50 μL of blocking buffer was mixed with 10 μL of 10% PEG 8000 and allowed to react for 5 minutes. The entire volume was then added to the lateral flow strip construct. The virus-nanoparticle complexes that migrated to the capture zone resulted in a positive reaction. The control biotin-conjugated nanoparticles that migrated past the test capture zone, irrespective of the presence of virus-nanoparticle conjugates, were arrested at the control capture zone of the device, thus confirming the integrity of the device. The arrangement of the test strip was similar to the illustrated diagram in FIGS. 1 and 2 with test and control lines. The solid phase capture particles in the reaction chamber were desiccated polystyrene microparticles.

The lateral flow desiccated assembly consisted of an absorbent medium containing 100% cotton linter Grade #237 (Ahlstrom Paper Group). The conjugate medium consisted of 8975 borosilicate glass fiber with the water-soluble synthetic polymer polyvinyl alcohol (PVA) binder (Whatman). Control biotin-BSA conjugated red nanoparticles were impregnated into the conjugate medium. The nitrocellulose medium was 25 mm wide HiFlow Plus HF09004 (Millipore) with a test capture zone with 1.0 mg/mL specific anti-virus antibody and a control capture zone containing 1.0 mg/mL NeutrAvidin”. Both the capture zones were striped as lines using IVEK Digispense 2000. The nitrocellulose membranes were blocked with StabilCoat™ (SurModics; Cat#SC02-1000) buffer for 30 min. An absorbent consisted of #470 (Whatman) was used to facilitate flux. The media were mounted in the typical fashion on an adhesive support consisting of 0.01″ White Polystyrene with PSA adhesive (GL 187; G&L Precision Die Cutting, Inc.). Test device with all the compartments were completely assembled before testing.

### Example 2

### Non-Specific Attachment of Nucleic Acid Followed by Amplification And Detection

In one method, the immobilization of a DNA and/or RNA is achieved through the use of solid-phases capable of binding nucleic acid targets after extraction from a sample (Gerdes et al. 2004, Mondesire et al. 2000). Under optimal buffer conditions, the capture of the nucleic acid targets occurs immediately after extraction and the nucleic acid captured using this binding material can be amplified directly on the solid phase using a variety of amplification strategies. This approach has been shown to be capable of detecting single copies of genes from as little as 10 μl of blood. Several gene targets have been tested by this method.

In the preferred embodiment, we envision the binding of amplicon on a solid phase capable of mobility after reconstitution. This is followed by the migration or deposition of amplicon-particle complexes to a site on the membrane for specific capture and detection.

The use of nanoparticles in this fashion will greatly increase the surface area of bound molecular targets to levels that can be easily detected with standard diagnostic tools.

### REFERENCES

Gerdes, J. G., Mondesire, R. R. and Hansen, L. 2004. Lateral flow system for nucleic acid detection. US Published Patent Application 20040110167
Mondesire, R. R., Kozwich, D. L., Johansen, K. A., Gerdes, J. C. and Beard, S. E. 2000. Solid-phase nucleic acid extraction, amplification and detection in molecular diagnostics. May/June Issue. IVD Technology Supplement. Canon Communications LLC 9-13.

1. A device in which non receptor-mediated attachment of an analyte to microparticles, nanoparticles or other binding surfaces is achieved, followed by migration or transfer of the analyte-bound complexes to another zone on the device where specific capture occurs

2. The device of claim 1, wherein the initial attachment mechanisms of target molecules are not due to specific ligand-receptor or antibody-antigen interactions

3. The device of claim 1, wherein the initial attachment exploits functional groups on the particles, nanostructures or other solid phases

4. The device of claim 1, wherein the particles range from nanometers to several microns in diameter

5. The device of claim 1, wherein the particles are gold colloid, metal sols, latex beads, microparticles or uniform microspheres

6. The device of claim 1, wherein the particles are immobilized, or in suspension

7. A method for the determination of the presence of an analyte in a sample wherein binding entities, if immobilized, are capable of reconstitution followed by migration or transfer to a capture zone

8. The method of claim 7, wherein multiple particle binding types and particle colors are used for multiple analyte detection

9. The method of claim 7, wherein the analyte comprises compounds, molecules and various targets used in diagnostics tests

10. The method of claim 7, wherein the analyte comprises any material of biological significance such as material derived from plants, animals and water

11. The method of claim 7, wherein the analyte consists of nucleic acid targets
